(19) United States
(12) Patent Application Publication
(10) Pub. No.: US 2016/0304924 A1
(43) Pub. Date: Oct. 20, 2016

(54) SYNERGISTIC FOOD FERMENTATION UTILIZING FUNGAL MYCELIUM AND BACILLI

(71) Applicants: Kevin Henry Fortin, Williams, IN (US); John Favier, Boulder, CO (US); Mara Jane King, Boulder, CO (US)

(72) Inventors: Kevin Henry Fortin, Williams, IN (US); John Favier, Boulder, CO (US); Mara Jane King, Boulder, CO (US)

(21) Appl. No.: 15/098,119

(22) Filed: Apr. 13, 2016

Related U.S. Application Data
(60) Provisional application No. 62/148,159, filed on Apr. 15, 2015.

(51) Int. Cl. C12P 19/04; A23B 9/28

(52) U.S. Cl. CPC C12P 19/04; A23L 1/105; A23B 9/28; A23V 2002/00; A23Y 2220/00

(57) ABSTRACT

Foods that are fermented with synergistic and simultaneous use of bacilli and mycelial cultures. The foods include a cellulosic food substrate containing detectable amounts of 1,3/1,6 β-glucan which has been fermented by a bacilli strain of bacteria in cooperation with mycelium of a strain of Agaricomycetes fungi including polyporales. The bacilli strain maintains the food substrate at a pH of less than 4.7 to inhibit growth of pathogenic microbes and the consequent need to sterilize the food substrate in a sterile environment prior to fermentation by the mycelial culture.

### FIELD OF THE INVENTION

This invention pertains to fermentation of food substrates utilizing fungal mycelium, and particularly to synergistic fermentation of food substrates utilizing fungi and bacteria.

### BACKGROUND OF THE INVENTION

Fermented foods have existed since prehistory. In addition to preserving food, fermentation may improve nutrition, bioavailability of nutrients and modify flavor of a variety of foods. Recently scientific evidence shows that the microbiology utilized in fermentation can have significant health benefits for both humans and animals.

Cheese, for example, is made by fermenting milk. It's a preserved product that can have a variety of delicious flavors. Wine is a fermentation product of grape juice. Fermentation not only enables preservation of the grape juice for years, but also introduces a flavor complexity that many appreciate.

Vegetables are also fermented. Sauerkraut, pickles, kimchi and numerous other products have been traditionally fermented as a way of preserving the vegetables and also a way of modifying the flavor of vegetables.

Soy sauce is typically fermented with Aspergillus sojae and Aspergillus oryzae molds. These are Ascomcota mold and some species are pathogenic, creating aflatoxin. These molds grow quite well in non-sterile conditions. Aspergillus sojae are from the class of Fungi known as Eurotiomycetes.

Tempeh is typically fermented with spores from the mold Rhizopus oligosporus, or Rhizopus oryzae. These molds are lower fungi from the order of Mucorales. They grow quite well in non-sterile conditions typically found in food processing facilities.

### Fermentation Bacteria

Historically, four species of lactic acid bacteria (LAB) have been identified as organisms that are present in sauerkraut fermentations: Leuconostoc mesenteroides, Lactobacillus brevis, Pediococcus pentosaceus, and Lactobacillus plantarum. In one embodiment of the invention these are combined in nutrient dense solution to create a bacillius ferment starter.

Yoghurt is fermented using Lactobacillus delbrueckii subspecies bulgaricus (l. d. bulgaricus) and Streptococcus thermophilus bacteria. In addition, other lactobacilli and bifido bacteria are also sometimes added during or after culturing yogurt. Yoghurt fermented with the l. d. Bulgaricus is recognized for providing health benefits in humans including immuno-balancing and longevity enhancing properties. In an alternate embodiment of the invention these are combined in nutrient dense solution to create a bacillius starter liquid.

Lactobacillus brevis, Lactobacillus delbrueckii subspecies bulgaricus and Lactobacillus plantarum are a species of the genus Lactobacillus.

These are recognized as being part of the class Bacilli, order Lactobacillales, family Lactobacillaceae and genus Lactobacillus. In another alternate embodiment of the invention various Lactpbacillaceae are combined in nutrient dense solution to create a bacilli starter liquid.

Generally fermentation with Bacilli increases the acidity (decreases the pH) of the food substrate and thus inhibits pathogenic contamination of the substrate.

Mushroom mycelium has been used for fermented chocolate, coffee and grains. One historical drawback of fermentation with mushrooms is that these fungi grow more slowly than bacteria. They generate a macroscopic mycelial structures that secrete myco-enzymes. It typically takes more time to grow mycelium than simple replication of most bacteria. As a result, strict conditions of sterility have been maintained to inhibit competition by pathogenic microorganisms such as lower fungi, pathogenic bacteria and particularly molds.

What is desired is a way to inhibit pathogenic microbial contamination of foods to enable fermentation by mushroom mycelium. What is also desired is a way of accomplishing fermentation that is inexpensive, and that can be accomplished in a standard food-processing setting. What is also desired is a way of fermenting foods that does not require a clean-room setting.

### SUMMARY OF THE INVENTION

The present invention includes a food substrate that is synergistically fermented by mushroom mycelium and a strain of Bacilli and methods thereof. The fermented food substrate can be animal feed, or food for human consumption.

In one embodiment the food substrate is processed using a ferment starter including an active bacillus strain of bacteria and active mycelium of a strain of white rot fungi and detectable amounts of 1,3/1,6 β-glucan. The bacillus strain maintains the solution at a pH of less than 4.7 to inhibit growth of pathogenic microbes. The fungi delivers detectable amounts of 1,3/1,6 β-glucan to the food substrate to enhance immune function to the consumer of the food substrate. In the case of animal feed, the present invention when fed to livestock can improve immune function of the livestock. This minimizes the need to use antibiotics in the food substrate to maintain herd or flock immunity. Optimally, the present invention would eliminate the need for prophylactic feeding of antibiotics. In both humans and livestock the food substrate becomes a functional food to improve immune function, reduce inflammation and yield better metabolic function.

Livestock in an embodiment of the invention includes cattle, poultry, goats, sheep, and swine, as well as farm-raised marine livestock such as fish, krill, shrimp and other farm-raised marine animals. The food substrate includes corn, wheat, barley, rye, rice, and other grains in an embodiment of the invention.

The ferment starter is either a liquid in solution, or a dried product.

The dried ferment starter is dried at low temperature to a moisture content effective to inhibit growth of pathogenic microbes but not high enough to deactivate the ferment starter. Preferably the moisture content is less than 15%, and more preferably less than 8%.

The bacilli strain is preferably a bacillius strain selected from the group consisting of, Leuconostoc mesenteroides, Lactobacillus brevis, Pediococcus pentosaceus, Lactobacillus plantarum, and combinations thereof. The bacilli strain can be mixed with various other strains of bacteria commonly utilized in fermentation or acidification of foods.

In one embodiment the mycelium is from the class Agaricomycetes and order Polyporales. In another embodiment of the invention the mycelium is from the class Agaricomycetes and the division of Ascomycota, class Sordaiomycetes and family of Cordycipitaceae. In yet another embodiment of the invention, the mycelium is from the class Agaricomycetes and from the order Russulales and family Hericiaceae and the starter contains detectable amounts of Erinacine. In another embodiment, the mycelium is from the class Agaricomycetes and from the order Polyporales, family Ganodermataceae, and the species Ganoderma Lucidum and the starter contains detectable amounts of metabolites from Ganoderma Lucidium mycelium.

It can be appreciated that combinations of the mycelium cultures can be used in accordance with the present invention. Those utilized for the starter can be added separately to a food substrate or mixed with the starter and mixed with the food substrate.

The present invention also includes a fermented food including a food substrate containing detectable amounts of 1,3/1,6 β-glucan which has been fermented by a bacilli strain of bacteria in cooperation with mycelium of a strain of Agaricomycetes fungi. The bacilli strain maintains the food substrate at a pH of less than 4.7 to inhibit growth of pathogenic microbes.

The food substrate is a cellulosic food substrate selected from the group consisting of vegetables, whole grains, processed grains, rice bran, wheat middlings, and combinations thereof. It can be appreciated that the food substrate can be pro-biotic, i.e. containing live cultures, or the cultures can be deactivated by heat or other means. Pro-biotic foods have shown to support immune system function in vivo.

The combined effect of pro-biotic foods with mushroom derived 1,3/1,6 β-glucan offers superior immune function support.

The present invention further includes a pro-biotic beverage including water, an active bacilli strain of bacteria and fungal metabolites from an Agaricomycetes fungi. In various embodiments, the metabolites include detectable amounts of 1,3/1,6 β-glucan and ganoderic acid, Erinacine, or combinations thereof.

In another embodiment, a fermented food product includes rice bran or wheat middlings simultaneously fermented with a combination of bacillus and mycelium of a white rot fungi. In a variation of this embodiment, the rice bran is toasted or roasted prior to fermentation to improve flavor. In an alternate embodiment, the rice bran is raw prior to fermentation and the cooperation of the bacillus and the mycelium of the white rot fungi inhibits growth of pathogenic microbes. Preferably, the rice bran has a pH of less than 4.7.

The synergistic fermentation of the present invention can be accomplished sequentially, first with action of the Bacilli culture, and then with the action of the mycelium culture. In another embodiment of the invention simultaneous fermentation is accomplished.

### BRIEF DESCRIPTION OF THE DRAWING

FIG. 1 is a flow chart of a method in accordance with the present invention.

### DETAILED DESCRIPTION

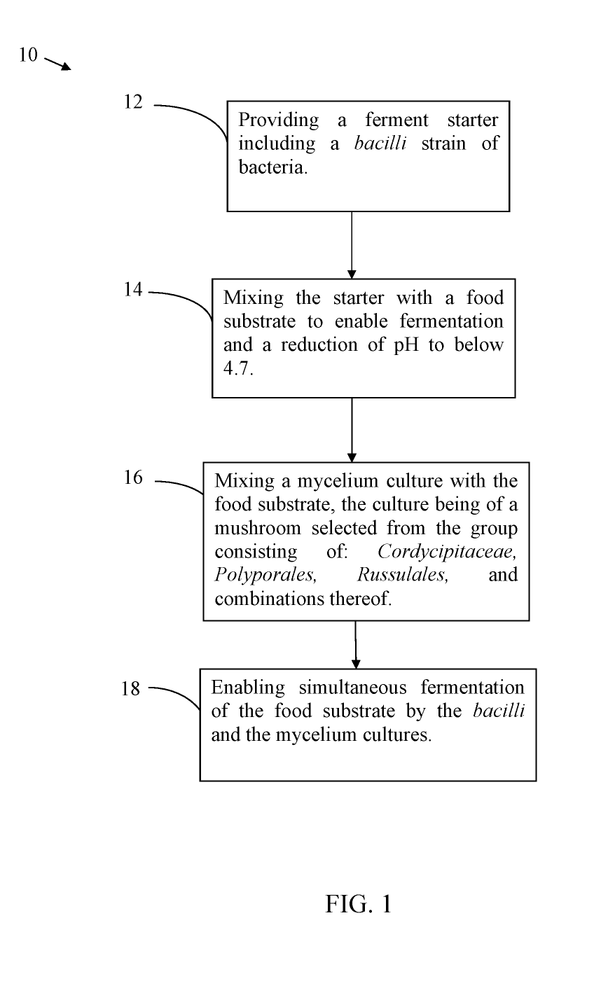

FIG. 1 shows a method 10 in accordance with the present invention. The method 10 includes the step 12 of providing a ferment starter including a Bacilli strain of bacteria. Next the step 14 mixes the ferment starter with the food substrate. The step 14 includes adding water and maintaining an ambient temperature of between 70° F.-78° F.

The step 14 of mixing the starter with the food substrate enables fermentation of the food substrate. This fermentation reduces the pH of the food substrate to below 4.7. Preferably the pH is reduced to 3.5-3.7. The step 14 acidifies the substrate which eliminates any likelihood of contamination of the substrate by pathogenic microbes.

Next the step 16 provides a mycelium culture and mixes the mycelium culture with the acidified food substrate. The mycelium culture is selected to endure acidic environments and can tolerate a range of pH levels of at least between 3.2 to 7.

The step 16 selects the mycelium culture from the group consisting of Cordycipitaceae, Polyporales, Russulales, and combinations thereof. More particularly, white-rot from the above groups are chosen due to the enzymatic characteristics of white rot fungi including the production of monooxygenase is and lignin modifying enzymes including laccase. Preferably the mycelium culture is chosen from well-known and well-accepted culinary or medicinal mushrooms.

The present invention includes simultaneous fermentation of a food substrate by both Bacilli and mycelium cultures. The mycelium culture, in one embodiment is from Polyporales fungi. The Bacilli functions to rapidly resist pathogenic microbes to enable mycelial growth of the fungi. The presence of both hasten the fermentation process.

Recent methods of fermentation have been devised utilizing mycelium of medicinal and culinary mushrooms. These mushrooms loosely fall in the phylum Basidiomycota, more particularly in the class Agaricomycetes and order Polyporales. Many culinary and medicinal mushrooms fall into the category of Polyporales. The mushrooms utilized in accordance with the present invention have mycelium capable of producing 1,3/1,6 β-glucan, which is understood to have superior bio-activity, and is produced in higher concentrations, than 1,3/1,4 β-glucan found in grains such as oats and barley.

An array of species of Cordyceps mushrooms have documented medicinal value and fall under the general division of Ascomycota, class Sordaiomycetes and family of Cordycipitaceae. In one embodiment of the invention Cordyceps mycelium is used with the Bacilli.

Hericium einaceus (aka Lions Mane Mushroom) is an Agaricomycetes from the order Russulales and family Hericiaceae. It has a unique aroma and delicious flavor. It produces unique metabolites including Erinacine which may promote nerve growth factor in humans. Both pro-biotic content due to the Bacillus and Erinacine cooperate to make a functional food that may improve cognative function and neuro-health.

Lions Mane also produces powerful anti-oxidants including threitol, D-arabinitol and palmic acid. Lions Mane metabolites my also regulate blood lipid levels and mad reduce blood glucose levels, as well as use in treatment of esophageal carcinoma. In one embodiment of the invention Lions Mane mycelium is used with the Bacilli.

In one embodiment, the mycelium is from the class Agaricomycetes and from the order Polyporales, family Ganodermataceae, and the species Ganoderma Lucidum and the starter contains detectable amounts of β-glucan, ganoderic acids, coumarin and mannitol, or combinations thereof. In a variation of this embodiment, the ganoderic acids are fungal metabolites of the mycelium. In an alternate embodiment, the ganoderic acids are produced by the fruiting body of Ganoderma Lucidium and added to the starter, or a fermentable substrate, or a fermented substrate.

The functional term “white rot fungi” includes the above-referenced mushrooms that produce a white colored mycelium and excrete enzymes that are particularly good at reducing cellulose and plant lignin.

The various Lignin-Modifying Enzymes (LME's) produced by Basidiomycota, particularly those from the class of Agaricomycetes, and more particularly, Polyporales, include a set of hydrolytic enzymes, namely endoglucanase, cellobiohydrolase and β-glucosidase. LME's are known to have the ability to modify a broad array of substrates.

Laccase, manganese-dependent peroxidase (MnP), lignin peroxidase (LiP), and versatile peroxidase (VP) are examples of well-known LME's. These LME's are capable of modifying plant lignin and many other organic materials.

One benefit of the above fungi and their use in fermentation that the mycelium of these fungi produce 1,3/1,6 β-glucan and other very beneficial fungal metabolites that show promise for improving human and animal health.

The present invention proposes a synergistic growth of Bacilli bacteria and fungi. There are numerous benefits of a combined fermentation culture.

One benefit is that the combined enzymatic action of both the fungi and the Bacilli cooperate to accelerate fermentation of many substrates. Another benefit is improved flavor and aroma of the fermented food substrate. A further benefit is a broader array of nutrients are produced. Another benefit is the bio-availability of nutrients is enhanced.

Action of the Bacilli inhibits contamination by pathogenic microorganisms including competing bacteria and molds. This is due at least in part to the ability of the Bacilli to lower the pH of the substrate material to below 6, and preferably below 4.7 to inhibit pathogenic microbiological growth. Optimal pH is between 3.5 and 4.6.

Action of the fungi is typically not inhibited by an acidic pH of the substrate and a pH of 4.7 is well within the range of the synergistic fungi proposed by the present invention. Strain selection improves performance of the fungi at the pH range of less than 4.7, and preferably below 3.7, or within the range of 3.5 to 3.7, which is a characteristic pH of many traditional ferments.

A further benefit of the synergistic action of the Bacilli bacteria and fungi is that food-grade sterility conditions are sufficient to enable action of the fungi. Food-grade sterility is defined herein as less than 35,000,000 particles per cubic meter of air in the size range of 0.5 microns and larger in diameter.

The ability to ferment foods using the fungi described herein to enhance flavor, add β-Glucans and other fungal metabolites, represents a leap forward in the art and enables the production of fermented food with improved functional properties at a much lower cost. As a result any food substrate, including vegetables, beans, coffee, cocoa, dairy products, grains, milling byproducts such as wheat middlings and rice husks, and various mixed animal feed, can be infused with the health promoting attributes of fungal metabolites at a commercially viable cost.

### Fungal Culture Production

Fungal mycelium can be grown on various substrates and in various environment in accordance with the present invention. Preferably, the fungal mycelium is cultivated on a medium such as a cellulosic substrate. The cellulosic substrate may include micro cellulose crystals, sawdust, wood pellets, or wood dowels. More preferably, the cellulosic substrate includes rice husks or wheat middlings.

The cellulosic substrate is mixed with the food substrate to enable solid state fermentation of the food substrate. The cellulosic substrate is separated from the food substrate after fermentation reaches a desired degree.

In an alternate embodiment, the cellulosic substrate is washed and agitated to remove microscopic sized particles of mycelium from the solid-state medium to yield a liquid-state mycelium culture.

In another embodiment, the fungal mycelium is cultivated in a liquid-state in a nutrient dense solution to yield a liquid-state mycelium culture.

Various examples below implement the present invention.

### Exemplary Products

The present invention includes a ferment starter including both a bacillus and mycelium culture. The starter can be liquid, or dried at low temperatures to maintain the bacilli and mycelium cultures in a viable form. This makes a ferment starter that can be sold commercially.

The present invention enables the simultaneous fermentation of a food substrate by both bacilli and mycelium cultures.

It can be appreciated that bacilli and mycelium cultures can be utilized at different times and for different durations for fermenting particular substrates to optimize flavor, β-glucan production, or other metabolite production.

In one embodiment, the bacillus is added to a substrate first, and after a desired period the mycelium can be added. This enables the pH of the substrate to become acidic, which inhibit pathogenic microbial growth and prepares the substrate for inoculation by the mycelium. In this embodiment, both the mycelium and the bacillus propagate simultaneously with synergy.

The present invention encompasses a wide range of food products, including snack bars, noodles, breads, fermented vegetables, fermented grains, beverages and numerous other food products. Each includes an ingredient produced in accordance with the present invention.

Grains and grain byproducts including wheat middlings, soybean hulls, and rice husks can be fermented in accordance with the present invention. In one embodiment the byproducts are ground to a granular form such as corn meal, rice bran or wheat bran prior to fermentation. In another embodiment, the byproducts are pelleted prior to fermentation.

It can be appreciated that the list of bacteria and fungi presented here is not exhaustive. The present invention is intended to include utilizing a non-pathogenic bacteria to enable fungal fermentation to produce fungal metabolites in a non-sterile environment. It can be appreciated that a variety of bacteria can be used including those that rapidly reduce the pH of a substrate to inhibit growth of pathogenic bacteria, molds and undesirable fungi. Further, the two fermentation processes can occur simultaneously, or sequentially.

The pre-processing of the food substrate is useful for many reasons. In the case of rice bran, the rice husk is often a byproduct of making white rice. The husk is separated and contains optimal amounts of cellulosic fiber to enable the efficient production of 1,3/1,6 β-glucan. Toasting the rice bran pasteurizes or sterilizes the rice bran to inhibit contamination by pathogenic microbes. The cooperation of toasting rice bran and applying the bacilli fermentation works well to inhibit microbial contamination. In this way, the process of producing 1,3/1,6 β-glucan can be accomplished under non-sterile conditions.

The pre-processing corn by steam rolling the whole corn into flakes, also pasteurizes or sterilizes the corn to inhibit contamination by pathogenic microbes. The cooperation of flaking the corn and applying the bacilli fermentation works well to inhibit microbial contamination. In this way, the process of producing 1,3/1,6 β-glucan can be accomplished under non-sterile conditions.

Various examples of the present invention are provided:

### EXAMPLE 1

1 oz of bacilli starter liquid added to:
1 lb of shredded cabbage in one gallon of water
1 oz of fresh crushed garlic
1 oz of shredded ginger
1 oz of liquid-state mycelium culture from ganoderma lucidium.
   Allow to ferment for 1-3 weeks in a vented container.
   Can or bottle for distribution.

### EXAMPLE 2

1 oz of bacilli starter liquid added to:
1 lb of rice bran in two quarts of water.
1 oz each of salt, chiles, ginger, kelp, dried shitake.
1 oz of a cellulose medium infused with a mycelium culture from Hericium einaceus.
   Allow to ferment for 1-3 weeks.

The substrate (rice bran) can be propagated by mixing nutrients, more rice bran and more water. This yields a rice bran containing mushroom β-glucan. The fermented rice bran ran can be made into a snack bar, granola, animal feed, or other food product. In this example, the substrate can optionally be pretreated by roasting or toasting or steam cooking, to enhance flavor and reduce concentrations of pathogenic microbes found in the rice bran.

### EXAMPLE 3

A bacilli starter liquid including a mixture of least one lactobacillus strain and mycelium from an edible white rot fungi.

### EXAMPLE 4

1 oz of bacilli starter liquid and 1 oz of mycelium culture added to:
1 lb of rice bran in two quarts of water.
   Allow to ferment together to produce fermented rice bran.

### EXAMPLE 5

1 oz of bacilli starter liquid and 1 oz of liquid mycelium culture added to:
1 lb of toasted rice bran in one quarts of water;
   Agitate mixture periodically.
   Allow to ferment together at 75° F. for 1-3 weeks produce fermented rice bran containing 1,3/1,6 β-Glucan.

### EXAMPLE 6

1 oz of bacilli starter liquid and 1 oz of liquid mycelium culture added to:
1 lb of toasted rice bran in one gallon of water;
   Allow to ferment together at 75° F. for 1-3 weeks produce fermented rice bran containing 1,3/1,6 β-Glucan.
   Use the fermented rice bran to make bread, a snack bar, a breakfast cereal, or cracker containing 1,3/1,6 β-Glucan.

### EXAMPLE 7

1 oz of bacilli starter liquid added to:
1 lb of toasted rice bran in one gallon of water;
   Allow to ferment at 75° F. for 1-3 days to produce fermented rice bran having a pH of less than 5.
   Mixing 1 oz of liquid mycelium culture of Ganoderma lucidium to the fermented rice bran.
   Allow to ferment for 1-3 weeks.
   Use the fermented rice bran to make bread, a snack bar, a breakfast cereal, or cracker containing 1,3/1,6 β-Glucan.

### EXAMPLE 7

1 oz of bacilli starter liquid added to:
1 lb of corn meal in one gallon of water;
   Allow to ferment at 75° F. for 1-3 days to produce fermented corn meal having a pH of less than 5.
   Mixing 1 oz of liquid mycelium culture of Ganoderma lucidium to the fermented corn meal.
   Allow to ferment for 1-3 weeks.
   Use the fermented corn meal to make livestock feed, bread, a snack bar, a breakfast cereal, or cracker containing 1,3/1,6 β-Glucan.

### EXAMPLE 7

1 oz of bacilli starter liquid added to:
1 lb of flaked corn, made by steaming and rolling whole corn, in one gallon of water;
   Allow to ferment at 75° F. for 1-3 days to produce fermented flaked corn having a pH of less than 5.
   Mixing 1 oz of liquid mycelium culture of Ganoderma lucidium to the fermented flaked corn.
   Allow to ferment for 1-3 weeks.
   Use the fermented flaked corn to make a livestock feed product containing 1,3/1,6 β-Glucan.

While the present invention has been disclosed by way of example, it should be appreciated that the processes, the food substrate, and the microbiological components can change to yield the result of a fermented food substrate containing 1,3/1,6 β-Glucan. For example, the acidification of the food substrate can be accomplished in many ways, for example submerging or treating the food substrate with an acid. The acid can be a vinegar, for example. Also, when Bacilli is used to acidify the food substrate, the Bacilli can be active or be de-activated when the fungal mycelium is added. Further, since the food substrate can be consumed by either livestock or people, the term “food” should be broadly interpreted to include food for any live organism.

1. A fermented food comprising:
a food substrate containing detectable amounts of 1,3/1,6 β-glucan;
the food substrate having been fermented by a bacilli strain of bacteria to maintain the food substrate at a pH of less than 4.7;
the food substrate also having been fermented by mycelium of a strain of Agaricomycetes fungi to produce detectable amounts of 1,3/1,6 β-Glucan.

2. The fermented food as set forth in claim 1 wherein, the food substrate is selected from the group consisting of whole grains, processed grains, rice bran, wheat middlings, corn and combinations thereof.

3. The fermented food as set forth in claim 1, wherein the food substrate is flaked corn made by steaming and rolling whole corn prior to fermentation.

4. The fermented food as set forth in claim 1, wherein the food substrate is toasted rice bran made by toasting rice bran prior to fermentation.

5. The fermented food as set forth in claim 1, wherein the fermented food has been simultaneously fermented with a combination of bacillus and mycelium of a mushroom selected from the group consisting of, culinary mushrooms, medicinal mushrooms, or combinations thereof.

6. A food product comprising:
wheat middlings fermented with bacilli bacteria and mycelium of a white rot fungi to contain detectable amounts of 1,3/1,6 β-glucan.

7. The food product as set forth in claim 6, wherein the wheat middlings have pH of less than 4.7.

8. Corn containing detectable amounts of 1,3/1,6 β-glucan comprising:
the corn having been subject to a first fermentation process by a bacilli strain of bacteria to inhibit growth of pathogenic microbes;
the corn having been subject to a second fermentation process by mycelium of a strain of Agaricomycetes fungi to produce 1,3/1,6 β-Glucan.

9. Corn as set forth in claim 8, wherein the first and second fermentation processes are sequential.

10. Corn as set forth in claim 8, wherein the first and second fermentation processes are simultaneous.
